(19) United States
(12) Patent Application Publication
(10) Pub. No.: US 2012/0020869 A1
(43) Pub. Date: Jan. 26, 2012

(54) Microporous and Mesoporous Carbon Xerogel Having a Characteristic Mesopore Size and Precursors Thereof and Also a Process for Producing These and Their Use

(75) Inventors: Christian Scherdel, Estenfeld (DE); Gudrun Reichenauer, Gerbrunn (DE)

(73) Assignee: Evonik Carbon Black GmbH

(21) Appl. No.: 13/002,230

(22) PCT Filed: Mar. 11, 2009
(86) PCT No.: PCT/EP09/52861

§ 371 Date: Sep. 21, 2011

Related U.S. Application Data
(60) Provisional application No. 61/070,892, filed on Mar. 26, 2008.

(30) Foreign Application Priority Data
Mar. 26, 2008 (DE) 10 2008 015 788.0

(51) Int. Cl. C01B 31/00; C01B 31/02; C07C 47/00

(52) U.S. Cl. 423/445 R; 568/420

(57) ABSTRACT

The invention relates to a microporous and mesoporous carbon xerogel and organic precursors thereof based on a phenol-formaldehyde xerogel. A characteristic parameter common to carbon xerogels is a peak in the mesopore size distribution determined by the BJH method (Barrett-Joyner-Halenda) from nitrogen absorption measurements at 77 K in the range from 3.5 nm to 4 nm. The production process is characterized firstly by the low starting material costs (use of phenol instead of resorcinol) and secondly by very simple and cost-effective processing; convective drying without solvent exchange instead of supercritical drying or freeze drying. The carbon xerogels and their organic phenol-formaldehyde xerogel precursors have densities of corresponding to a porosity of up to 89%, and the xerogels can also have a relevant mesopore volume. The carbon xerogels obtained from the phenol-formaldehyde xerogels are also microporous.

The invention provides a porous carbon xerogel with characteristic mesopore size and the precursor thereof in the form of a phenol-formaldehyde xerogel (PF xerogel), and also a process for production thereof by means of a sol-gel process with subcritical drying of the wet gel under standard conditions (room temperature and 1013 mbar). A typical feature of these phenol-formaldehyde-based carbon xerogels (=pyrolyzed PF xerogel) is a clearly identifiable peak in the pore size distribution by the BJH method (Barrett-Joyner-Halenda; DIN 66134) between 3.5 nm and 4.0 nm from measurements with nitrogen sorption at 77 K.

### STATE OF THE ART

Aerogels, cryogels and xerogels are employed in many fields. In principle, the materials mentioned differ by the type of drying method. Aerogels are defined by supercritical drying, cryogels by freeze-drying, and xerogels by convective subcritical drying under standard conditions.

Aerogels are a material whose morphological properties have very good adjustability; the spectrum of fields of use thereof is therefore wide. In the gas permeation or adsorption sector, aerogels can be used as a filter, gas separation layer or wastewater processor, or in chromatography. The mechanical and acoustic properties thereof recommend them as shock absorbers, meteorite bumpers or acoustic output adaptors. Aerogels are present in optics as IR reflectors or IR absorbers. Owing to their defined porosity, aerogels can be used as electrodes, dielectric layers or as a thermal insulation material. In addition, aerogels can be used as a support material or matrix in catalysts, or in medical components or sensors.

A great disadvantage of carbon aerogels and the organic precursors thereof has to date been the enormous costs, since expensive resorcinol was firstly required for the production, and the gel secondly had to be dried supercritically [1, 2]. In the last few years, numerous attempts have been made to reduce the costs. For example, in the case of xerogels, a solvent exchange has been carried out instead of supercritical drying, in order to replace the water with a liquid having lower surface tension (e.g. ethanol, acetone, isopropanol) (see, for example [3, 4]), and they were then dried under standard conditions. Attempts have also been made to replace the expensive resorcinol with less expensive starting materials, for example cresol [5]. The combination of phenol and furfural also leads in principle to homogeneous monolithic structures [6, 7], but furfural is firstly more expensive than formaldehyde, which counteracts the cost saving by the use of phenol, and the handling of furfural is secondly more problematic and not especially desirable in industrial scale production. There have also already been reports of porous carbons based on phenol-formaldehyde condensates [8, 9]. However, it has not been possible to dispense with complex drying processes such as freeze-drying or supercritical drying with solvent exchange.

A particular method which can be used for characterization of aerogels, xerogels and porous materials in general is the established nitrogen sorption analysis, since this allows a wide range of information about micro- and mesoporosity, and also pore size distribution, of the materials studied to be obtained.

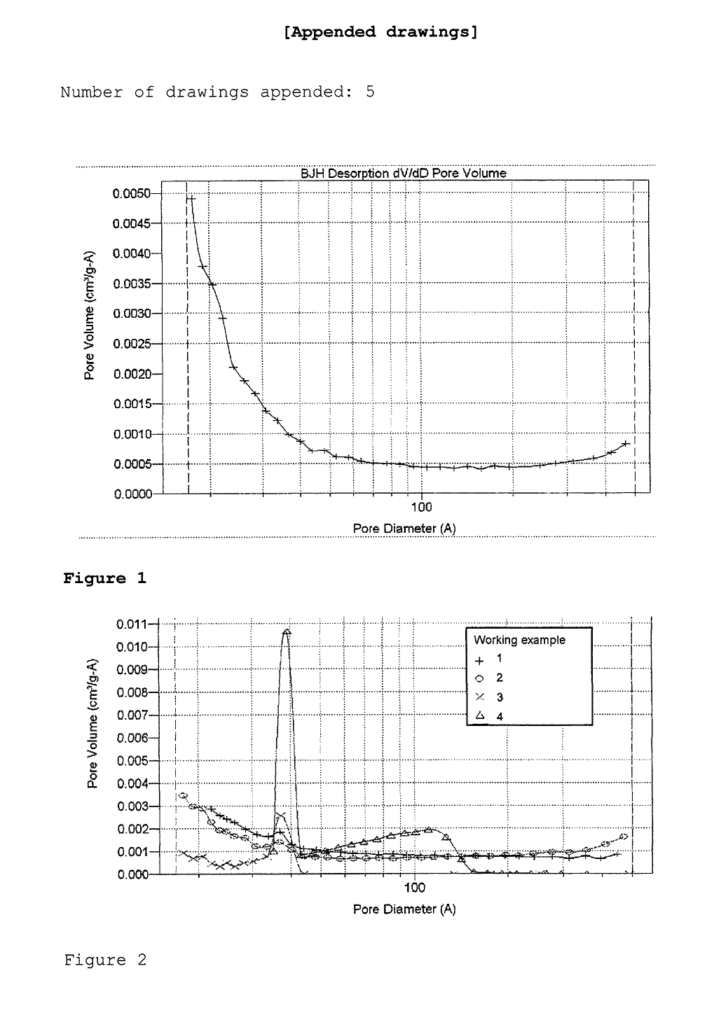

In the case of carbon aerogels in general, the pore size distribution can be varied within a relatively wide range as a function of the synthesis parameters and the production process; a characteristic recurrent parameter which is common to the carbon aerogels and xerogels and is independent of the synthesis parameters has not been observed to date. FIG. 1 shows the pore size distribution of a resorcinol-formaldehyde (RF)-based carbon xerogel. For the production, a molar ratio of resorcinol to the catalyst (Na₂CO₃) of 1300, a molar ratio of formaldehyde to resorcinol of 2 and a concentration of resorcinol and formaldehyde in the aqueous start solution of 30% was selected. The RF sample was processed with a gelation cycle; at room temperature, 50° C. and 90° C. for 24 h each. Subsequently, the wet gel was exchanged twice with acetone for 24 h each, then dried convectively, and the RF xerogel was finally converted at 800° C. under an oxygen-free protective gas atmosphere to the carbon xerogel, which was analyzed by nitrogen sorption.

An overview of the prior art in the conventional system composed of resorcinol and formaldehyde is given, for example, by the publications by Tamon et al. and Yamamoto et al. [10-12].

### OBJECT OF THE INVENTION

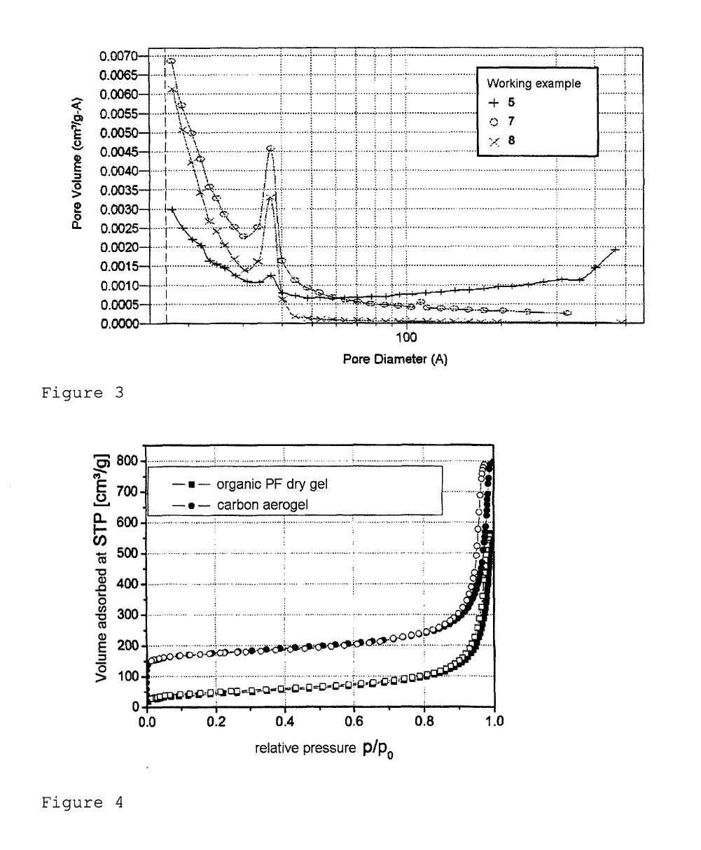

The object of the invention is a micro- and mesoporous carbon xerogel and the organic precursor thereof, said xerogel meeting the requirements on the performance properties of aerogels and xerogels in full, and additionally having a substance-specific property which distinguishes the inventive carbon xerogel from already known carbon aerogels and xerogels, for example based on resorcinol-formaldehyde. A common feature of the inventive carbon xerogels is a characteristic peak in the mesopore distribution between 3.5 nm and 4.0 nm by the BJH method (Barrett-Joyner-Halenda; DIN 66134), which is obtained from measurements with nitrogen sorption at 77 K (see FIG. 2 and FIG. 3).

It is a further object of this invention to provide a process for producing the carbon xerogels and the organic PF xerogel precursor thereof. The production process is characterized by the use of inexpensive reactants with a very simple and cost-effective process. The starting materials used are phenol, especially the inexpensive monohydroxybenzene, and formaldehyde, which are crosslinked with a catalyst (acid or base) and a solvent (alcohol, ketone or water), by means of the sol-gel process. The use of the costly resorcinol (1,3-dihydroxybenzene) is completely dispensed with. Furthermore, the process detailed here enables the production of xerogels of low density and high micro- and mesoporosity without the complex process steps of freeze-drying or supercritical drying. In addition, a solvent exchange is not necessary in the present invention.

The two reactants, phenol and formaldehyde, react with one another in a sol-gel process. The solvent used is water or an alcohol, for example n-propanol; the catalysts used are either acids or bases, for example hydrochloric acid (HCl) or sodium hydroxide (NaOH). Once the sol-gel process has ended and a monolithic wet gel has formed, the gel, without further aftertreatment, can be dried by simple convective drying at room temperature or at elevated temperature (e.g. 85° C.) The mechanically stable wet gel precursor can prevent collapse of the gel network. By pyrolysis of the organic PF-xerogel precursor at temperatures above 600° C. under an oxygen-free protective gas atmosphere, a monolithic carbon xerogel is obtained.

The resulting monolithic carbon xerogels and the organic PF xerogel precursors thereof have densities of 0.20-1.20 g/cm³, which corresponds to a porosity of up to 89%. In addition, the carbon xerogels and the organic PF xerogel precursors thereof have a mesoporosity by the BJH method of up to 0.76 cm³/g.

For specific applications of the xerogels in powder form, for example as IR absorbers, the monolithic PF xerogels or carbon xerogels can be comminuted to the desired size by customary grinding methods.

### EXAMPLES

### Working Example 1

In a beaker, 3.66 g of phenol are mixed with 6.24 g of formaldehyde solution (aqueous 37% formaldehyde solution stabilized with approx. 10% methanol) and 26.27 g of n-propanol (corresponds to a molar ratio of formaldehyde to phenol of F/P=2 and a concentration of the phenol and formaldehyde reactants in the mass of the overall solution of M=15%). The solution is stirred on a magnetic stirrer until the phenol has dissolved completely. Subsequently, 3.83 g of 37% HCl are added (corresponds to a molar ratio of phenol to the catalyst of P/C=1). The solution is then introduced into a beaded edge bottle of height 10 cm (diameter 3 cm), and the beaded edge bottle is sealed airtight. The beaded edge bottle together with the sample is heated at 85° C. in an oven for 26 hours.

After 26 hours, a monolithic organic wet gel is obtained, which is subsequently dried convectively at 65° C. in a drying oven for 70 hours. A monolithic organic PF xerogel is obtained with a macroscopic density of 0.37 g/cm³. The organic PF xerogel is converted by pyrolysis at 800° C. under an argon atmosphere to a carbon xerogel. The carbon xerogel thus obtained has a macroscopic density of 0.42 g/cm³, a modulus of elasticity of 8.41*10⁸ N/m², a specific electrical conductivity of 2.4 S/cm, a specific surface area of 515 m²/g (by BET method, DIN ISO 9277:2003-05), a micropore volume of 0.16 cm³/g (by T-plot method, DIN 66135-2), an external surface area of 138 m²/g and a mesopore volume of 0.37 cm³/g (DIN 66134).

### Working Example 2

In a beaker, 6.11 g of phenol are mixed with 10.39 g of formaldehyde solution (aqueous 37% formaldehyde solution stabilized with approx. 10% methanol) and 21.38 g of n-propanol (corresponds to F/P=2; M=25%). The solution is stirred on a magnetic stirrer until the phenol has dissolved completely. Subsequently, 2.18 g of 37% HCl are added (corresponds to P/C=2.95). The solution is then introduced into a beaded edge bottle of height 10 cm (diameter 3 cm), and the beaded edge bottle is sealed airtight. The beaded edge bottle together with the sample is heated at 85° C. in an oven for 24 hours.

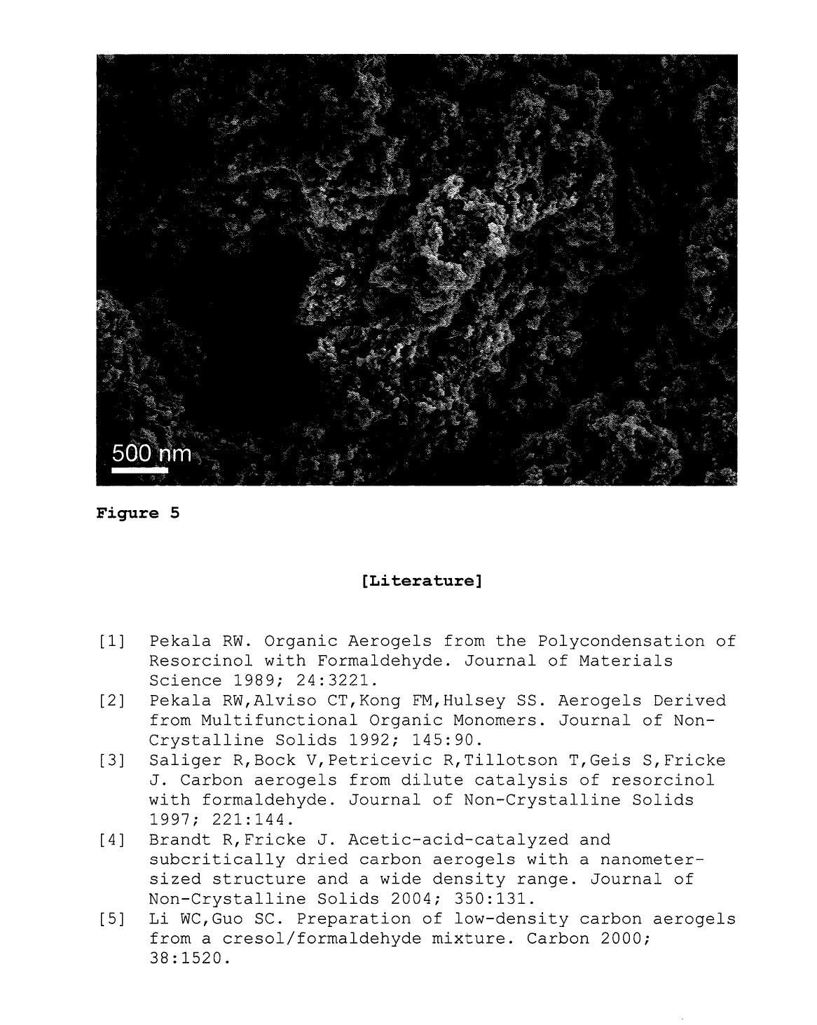

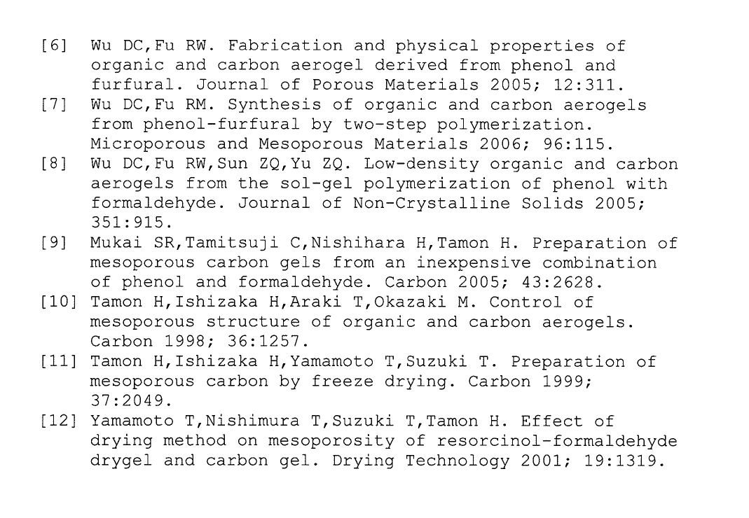

After 24 hours, a monolithic organic wet gel is obtained, which is subsequently dried convectively at 65° C. in a drying oven for 72 hours. This gives a monolithic, ochre-colored, organic PF xerogel with a macroscopic density of 0.48 g/cm³. The evaluation of the sorption isotherm from FIG. 4 gives a specific surface area (BET surface area) of 157 m²/g, an external surface area of 130 m²/g and a mesopore volume of 0.38 cm³/g. The organic PF xerogel is converted to a carbon xerogel by pyrolysis at 800° C. under an argon atmosphere. The carbon xerogel thus obtained has a macroscopic density of 0.54 g/cm³, a specific surface area (BET) of 657 m²/g, a micropore volume of 0.21 cm³/g, an external surface area of 150 m²/g and a mesopore volume of 0.76 cm³/g (see also sorption isotherm in FIG. 4). A scanning electron microscope (SEM) image (FIG. 5) shows a nanoscale morphology typical of carbon aerogels and xerogels. Elemental analysis of the carbon sample by means of EDX (energy-dispersive X-ray spectroscopy) shows, in the carbonized state of the xerogel, high-purity carbon with only low proportions of oxygen.

### Working Example 3

In a beaker, 6.11 g of phenol are mixed with 3.89 g of paraformaldehyde and 27.87 g of n-propanol (corresponds to F/P=2; M=25). The solution is stirred on a magnetic stirrer until the phenol and the paraformaldehyde have dissolved completely. Subsequently, 2.14 g of 37% HCl are added (corresponds to P/C=3). The solution is then introduced into a beaded edge bottle of height 10 cm (diameter 3 cm), and the beaded edge bottle is sealed airtight. The beaded edge bottle together with the sample is heated at 85° C. in an oven for 24 hours.

After 24 hours, a monolithic organic wet gel is obtained, which is subsequently dried convectively at 65° C. in a drying oven for 96 hours. This gives a monolithic organic PF xerogel with a macroscopic density of 1.00 g/cm³. The organic PF xerogel is converted to a carbon xerogel by pyrolysis at 800° C. under an argon atmosphere. The carbon xerogel thus obtained has a macroscopic density of 1.14 g/cm³, a specific surface area (BET) of 256 m²/g, a micropore volume of 0.10 cm³/g, an external surface area of 13 m²/g and a mesopore volume of 0.03 cm³/g.

### Working Example 4

In a beaker, 5.34 g of phenol are mixed with 9.09 g of formaldehyde solution (aqueous 37% formaldehyde solution stabilized with approx. 10% methanol) and 19.45 g of n-propanol (corresponds to F/P=2; M=25). The solution is stirred on a magnetic stirrer until the phenol has dissolved completely. Subsequently, 1.12 g of 37% HCl are added (corresponds to P/C=5). The solution is then introduced into a beaded edge bottle of height 10 cm (diameter 3 cm), and the beaded edge bottle is sealed airtight. The beaded edge bottle together with the sample is heated at 85° C. in an oven for 24 hours.

After 24 hours, a monolithic organic wet gel is obtained, which is subsequently dried convectively at room temperature for 5 days. This gives a monolithic organic PF xerogel with a macroscopic density of 0.99 g/cm³. The organic PF xerogel is converted to a carbon xerogel by pyrolysis at 800° C. under an argon atmosphere. The carbon xerogel thus obtained has a macroscopic density of 0.95 g/cm³, a specific surface area (BET) of 447 m²/g, a micropore volume of 0.17 cm³/g, an external surface area of 36 m²/g and a mesopore volume of 0.21 cm³/g.

### Working Example 5

In a beaker, 5.80 g of phenol, 0.31 g of 2,6-dimethylphenol, 10.39 g of formaldehyde solution (aqueous 37% formaldehyde solution stabilized with approx. 10% methanol) and 22.18 g of n-propanol are mixed (corresponds to F/P=2; M=25). The solution is stirred on a magnetic stirrer until the phenol and the 2,6-dimethylphenol have dissolved completely. Subsequently, 2.14 g of 37% HCl are added (corresponds to P/C=3). The solution is then introduced into a beaded edge bottle of height 10 cm (diameter 3 cm), and the beaded edge bottle is sealed airtight. The beaded edge bottle together with the sample is heated at 85° C. in an oven for 24 hours. After 24 hours, a monolithic organic wet gel is obtained, which is subsequently dried convectively at 65° C. in a drying oven for 96 hours. This gives a monolithic organic PF xerogel with a macroscopic density of 0.50 g/cm³. The organic PF xerogel is converted to a carbon xerogel under an argon atmosphere by pyrolysis at 800° C. The carbon xerogel thus obtained has a macroscopic density of 0.59 g/cm³, a modulus of elasticity of 19.7×10⁸ N/m², a specific surface area (BET) of 529 m²/g, a micropore volume of 0.17 cm³/g, an external surface area of 131 m²/g and a mesopore volume of 0.54 cm³/g.

### Working Example 6

In a beaker, 5.34 g of phenol, 9.09 g of formaldehyde solution (aqueous 37% formaldehyde solution stabilized with approx. 10% methanol) and 19.45 g of ethanol (denatured) are mixed (corresponds to F/P=2; M=25). The solution is stirred on a magnetic stirrer until the phenol has dissolved completely. Subsequently, 1.12 g of 37% HCl are added (corresponds to P/C=5). The solution is then introduced into a beaded edge bottle of height 10 cm (diameter 3 cm), and the beaded edge bottle is sealed airtight. The beaded edge bottle together with the sample is heated at 85° C. in an oven for 48 hours.

After 48 hours, a monolithic organic wet gel is obtained, which is subsequently dried convectively at room temperature for 96 hours. This gives a monolithic organic PF xerogel with a macroscopic density of 1.12 g/cm³. The organic PF xerogel is converted to a carbon xerogel by pyrolysis at 800° C. under an argon atmosphere. The carbon xerogel thus obtained has a macroscopic density of 1.04 g/cm³. The evaluation of the scatter curve obtained from small-angle X-ray scattering (SAXS) gives a micropore volume of 0.15 cm³/g.

### Working Example 7

In a beaker, 3.43 g of phenol, 17.52 g of formaldehyde solution (aqueous 37% formaldehyde solution stabilized with approx. 10% methanol) and 16.69 g of deionized water are mixed (corresponds to F/P=6; M=25). The solution is stirred on a magnetic stirrer until the phenol has dissolved completely. Subsequently, 2.37 g of 20% NaOH are added (corresponds to P/C=3.08). The solution is then introduced into a beaded edge bottle of height 10 cm (diameter 3 cm), and the beaded edge bottle is sealed airtight. The beaded edge bottle together with the sample is heated at 85° C. in an oven for 21 hours.

After 21 hours, a monolithic organic wet gel is obtained, which is subsequently dried convectively at room temperature for 72 hours. This gives a monolithic organic PF xerogel with a macroscopic density of 0.29 g/cm³ and with a modulus of elasticity of 1.67*10⁸ N/m². The organic PF xerogel is converted to a carbon xerogel by pyrolysis at 800° C. under an argon atmosphere. The carbon xerogel thus obtained has a macroscopic density of 0.20 g/cm³, an modulus of elasticity of 3.90*10⁸ N/m2, a specific surface area (BET) of 819 m²/g, a micropore volume of 0.30 cm³/g, an external surface area of 90 m²/g and a mesopore volume of 0.24 cm³/g.

### Working Example 8

In a beaker, 2.82 g of phenol, 20.31 g of formaldehyde solution (aqueous 37% formaldehyde solution stabilized with approx. 10% methanol) and 14.94 g of deionized water are mixed (corresponds to F/P=8; M=25). The solution is stirred on a magnetic stirrer until the phenol has dissolved completely. Subsequently, 2.37 g of 20% NaOH are added (corresponds to P/C=2.14). The solution is then introduced into a beaded edge bottle of height 10 cm (diameter 3 cm), and the beaded edge bottle is sealed airtight. The beaded edge bottle together with the sample is heated at 85° C. in an oven for 21 hours.

After 21 hours, a monolithic organic wet gel is obtained, which is subsequently dried convectively at room temperature for 72 hours. This gives a monolithic organic PF xerogel with a macroscopic density of 0.26 g/cm³ and with a modulus of elasticity of 0.085*10⁸ N/m². The organic PF xerogel is converted to a carbon xerogel by pyrolysis at 800° C. under an argon atmosphere. The carbon xerogel thus obtained has a macroscopic density of 0.25 g/cm³, an modulus of elasticity of 0.6*10⁸ N/m², a specific surface area (BET) of 619 m²/g, a micropore volume of 0.27 cm³/g, an external surface area of 6 m²/g and a mesopore volume of 0.08 cm³/g.

[List of Reference Numerals]

1. A mesoporous phenol-formaldehyde xerogel, wherein said mesoporous phenol-formaldehyde xerogel can be dried under standard conditions without exchange of solvent.

2. The phenol-formaldehyde xerogel as claimed in claim 1, wherein said mesoporous phenol-formaldehyde xerogel is pyrolyzed after drying and thus converted to a carbon xerogel.

3. The carbon xerogel as claimed in claim 2, wherein said carbon xerogel has a clearly identifiable peak in the pore size distribution by the BJH method (Barrett-Joyner-Halenda; DIN 66134) between 3.5 nm and 4.0 nm from measurements with nitrogen sorption at 77 K.

4. The carbon xerogel as claimed in claim 3, wherein said carbon xerogel is present in granule or powder form after a further treatment.

5. A process for producing a carbon xerogel, wherein a hydroxybenzene excluding resorcinol (1,3-dihydroxybenzene), especially monohydroxybenzene, 2,6-dimethylphenol, 2,4-di-tert-butylphenol and mixtures thereof, and formaldehyde gelate in a sol-gel process to give a wet phenol-formaldehyde gel, and then the wet gel is dried convectively at temperatures of 0° C.-200° C.

6. The process as claimed in claim 5, wherein the catalyst used is an acid or a base, especially hydrochloric acid (HCl) or sodium hydroxide (NaOH).

7. The process as claimed in claim 5, wherein the solvent is water, a ketone or an alcohol, especially n-propanol.

8. The process as claimed in claim 5, wherein the gelation is effected at temperatures of 20-120° C.

9. The process as claimed in claim 5, wherein there is no solvent exchange.

10. The process as claimed in claim 5, wherein the molar phenol to catalyst ratio P/C is between 0.1 and 30.

11. The process as claimed in claim 5, wherein the molar formaldehyde to phenol ratio F/P is between 0.5 and 20.

12. The process as claimed in claim 5, wherein the proportion by mass M of the phenol and formaldehyde reactants in the overall solution is between 5% and 60%.

13. The process as claimed in claim 5, wherein the PF xerogel is carbonized at more than 600° C. under a protective gas atmosphere.

14. The process as claimed in claim 13, wherein the carbon xerogel is activated at more than 500° C. with an oxygenous gas or a salt melt, or at a temperature below 200° C. with an acid or a base.

15. The process as claimed in claim 5, wherein the monolithic xerogel is comminuted into granules or powder, for example by the action of mechanical forces as in grinding.

16. The use of a xerogel corresponding to claim 1 as thermal insulation, an IR adsorber, a catalyst support, a filter, or as an electrode in supercapacitors, fuel cells or secondary cells, or for fluid or gas separation, or in sensor technology, or as an electrically and thermally conductive component in composites, or composite component in fiber-reinforced materials, or as casting molds for melts.

17. The use of a xerogel produced in claim 5, as thermal insulation, an IR adsorber, a catalyst support, a filter, or as an electrode in supercapacitors, fuel cells or secondary cells, or for fluid or gas separation, or in sensor technology, or as an electrically and thermally conductive component in composites, or composite component in fiber-reinforced materials, or as casting molds for melts.
